(19) United States
(12) Patent Application Publication
(10) Pub. No.: US 2020/0315529 A1
(43) Pub. Date: Oct. 8, 2020

(54) INFORMATION PROCESSING DEVICE, BIOLOGICAL INFORMATION MEASURING DEVICE, METHOD, AND NON-TRANSITORY RECORDING MEDIUM IN WHICH PROGRAM IS STORED

(71) Applicants: OMRON Corporation, Kyoto (JP); OMRON HEALTHCARE Co., Ltd., Kyoto (JP)

(72) Inventors: Toru DENO, Kyoto (JP); Naoki TSUCHIYA, Kyoto-shi, Kyoto (JP); Hiroshi USUI, Kyoto (JP); Kosuke INOUE, Kyoto (JP); Yoshiyuki MORITA, Kyoto-shi (JP); Yasushi MATSUOKA, Kyoto (JP)

(21) Appl. No.: 16/906,243

(22) Filed: Jun. 19, 2020

Related U.S. Application Data
(63) Continuation of application No. PCT/JP2018/046230, filed on Dec. 17, 2018.

(30) Foreign Application Priority Data
Dec. 27, 2017 (JP) 2017-251130

(51) Int. Cl. A61B 5/00; A61B 5/021; A61B 5/024; G16H 20/10; G16H 50/30

(52) U.S. Cl. CPC A61B 5/4848; A61B 5/021; G16H 50/30; G16H 20/10; A61B 5/024

(57) ABSTRACT

The present invention automatically acquires information about a living body at a time at which an effect of a medicine is generated. The present invention includes an acquisition control unit configured to acquire information about a medicine and time information about when the medicine is to be taken, a calculation control unit configured to calculate an effect generation time period in which an effect of the medicine is estimated to be generated, based on the information about the medicine and the time information, and a biological information acquisition control unit configured to perform processing for acquiring information about a living body, based on the effect generation time period.

### CROSS-REFERENCE TO RELATED APPLICATIONS

This is a continuation of International Application 2017-251130, with an international filing date of Dec. 27, 2017, and international Application PCT/JP2018/046230, with an international filing date of Dec. 17, 2018 filed by applicant, the disclosure of which is hereby incorporated by reference in its entirety.

### TECHNICAL FIELD

The invention relates to an information processing device, a biological information measuring device, a method, and a non-transitory recording medium in which a program is stored, including a support function related to medication and dosing.

### BACKGROUND ART

For example, in treatment of hypertension, generally, a physician prescribes an antihypertensive agent to a patient based on a consultation result, and the patient takes the prescribed antihypertensive agent, measures blood pressure in a determined time period every day by using a blood pressure monitor, and declares the measurement result to the physician in a next consultation.

Further, for example, a system for treating a patient at a remote place by using a network is also proposed. For example, in the system, a patient transfers vital measurement information such as blood pressure to a server via the Internet and registers the vital measurement information on a home page, and a physician views the vital measurement data registered on the home page of the server from a terminal of the physician and describes a comment for the patient, and this comment is notified to a vital measurement device of the patient at a next vital measurement time (see, for example, JP 2002-312487 A).

### SUMMARY OF INVENTION

However, in the consultation technique disclosed in JP 2002-312487 A, even in an office visit or in a remote consultation, a physician recognizes a treatment result of a patient with only a blood pressure value declared or measured by the patient as determination factors. Although the blood pressure value is important as the determination factors, a measurement time of the blood pressure value is not necessarily a time at which an effect of a medicine is generated. Therefore, there is a problem in that the blood pressure value declared or measured by the patient may not be appropriate as the determination factor.

The present invention has been made in view of the above-described circumstances, and an object thereof is to provide an information processing device, a biological information measuring device, a method, and a non-transitory recording medium in which a program is stored, capable of automatically acquiring information about a living body (hereinafter, also simply referred to as “biological information”) at a time at which an effect of a medicine is generated.

In order to solve the problems described above, the present invention adopts the following configuration.

In other words, an information processing device according to one aspect of the present invention includes an acquisition control unit configured to acquire information about a medicine and time information about when the medicine is to be taken, a calculation control unit configured to calculate an effect generation time period in which an effect of the medicine is estimated to be generated, based on the information about the medicine and the time information, and a biological information acquisition control unit configured to perform processing for acquiring information about a living body, based on the effect generation time period.

In the configuration described above, the effect generation time period in which the effect of the medicine is estimated to be generated can be calculated based on the information about the medicine and the time information about when the medicine is to be taken. Thus, the processing for acquiring biological information during the effect generation time period can be performed. Here, for example, a case where a “control signal for performing a measurement” is only output to the measurement unit that measures biological information or an “instruction that encourages a measurement” to a user is only presented is also included even when “acquisition” of the biological information is not performed. Therefore, the corresponding biological information can be automatically acquired in the effect generation time period in which the effect of the medicine is estimated to be generated, and thus it is possible to provide an information processing device capable of reliably acquiring information of how the medicine brings about changes in the living body.

Note that the time information is information related to time, and is not limited to only time and may include, for example, a day, a month, or a date.

The information processing device according to one aspect described above further includes a determination control unit configured to determine whether the medicine is effective on the living body, based on the information about the living body.

In the configuration described above, the biological information is acquired when the effect of the medicine is generated, and thus it is possible to determine whether the medicine is effective on the living body. Therefore, it is possible to provide an information processing device capable of helping with prescription of a medicine appropriate for a user.

The information processing device according to one aspect described above further includes an estimation control unit configured to estimate, from the information about the medicine and the information about the living body, whether the medicine has been taken.

In the configuration described above, a time at which the effect of the medicine is achieved can be recognized from the information about the medicine, and whether the medicine is taken at the time can be estimated with reference to biological information in the effect generation time period. Therefore, it is possible to provide an information processing device capable of supporting dosing of a medicine performed without being forgotten.

An information processing device according to one aspect described above includes an acquisition control unit configured to acquire information about a medicine and information about a living body, an estimation control unit configured to estimate, from the information about the medicine and the information about the living body, time information about when the medicine has been taken, a calculation control unit configured to calculate an effect generation time period in which an effect of the medicine is estimated to be generated, based on the information about the medicine and the time information, and a biological information acquisition control unit configured to perform processing for acquiring information about a living body, based on the effect generation time period.

In the configuration described above, a time at which the medicine has been taken can be estimated by observing a change in the biological information in response to the effect of the medicine, based on the information about the medicine and the biological information. Then, based on the information about the medicine and the time information about when the medicine is to be taken, the effect generation time period in which the effect of the medicine is estimated to be generated can be calculated. Thus, the processing for acquiring biological information during the effect generation time period can be performed. Therefore, the corresponding biological information can be automatically acquired in the effect generation time period in which the effect of the medicine is estimated to be generated, and thus it is possible to provide an information processing device capable of reliably acquiring information of how the medicine brings about changes in the living body.

The information processing device according to one aspect described above further includes a determination control unit configured to determine whether the medicine is effective on the living body, based on the information about the living body.

In the configuration described above, the biological information is acquired when the effect of the medicine is generated, and thus it is possible to determine whether the medicine is effective on the living body. Therefore, it is possible to provide an information processing device capable of helping with prescription of a medicine appropriate for a user.

In the information processing device according to one aspect described above, the determination control unit determines whether the medicine is effective on the living body by comparing first biological information measured at a time included in the time information with second biological information measured in the effect generation time period.

In the configuration described above, for example, the first biological information can be acquired at the time at which the medicine has been taken, and, furthermore, the second biological information can be acquired in the effect generation time period, and the first biological information when the effect is not yet generated and the second biological information when the effect is generated can be acquired. Whether the expected effect of the medicine is generated can be determined by comparing the two pieces of biological information.

Therefore, it is possible to provide an information processing device capable of supporting prescription of a medicine appropriate for a user.

In the information processing device according to one aspect described above, the determination control unit compares a first value indicated by the first biological information with a second value indicated by the second biological information, and makes a determination from a sign of a difference between the first value and the second value and a comparison result between an absolute value of the difference and a preset effective threshold value.

In the configuration described above, by comparing the first value indicated by the first biological information when an effect is not generated with the second value indicated by the second biological information in which the effect is expected to be generated, an increase or a decrease in value indicated by the biological information can be recognized by the sign of the difference between the values. Furthermore, a quantitative analysis of the effect of the medicine can be achieved by calculating the absolute value of the difference. Then, by comparing a preset effective threshold value by which the medicine is recognized as effective with the absolute value of the difference, whether the medicine taken is effective can be determined. Therefore, it is possible to provide an information processing device capable of supporting prescription of a medicine appropriate for a user.

The information processing device according to one aspect described above further includes a presentation control unit configured to present as a candidate for medication, when the determination control unit determines that the medicine is not effective, one or more medicines from a plurality of medicines including the medicine expected to have an effect.

In the configuration described above, when it is determined that the medicine taken is not effective, one or more medicines can be presented as a candidate for medication from a plurality of medicines including the medicine. Since a medical professional can select a medicine that is more likely to be effective by viewing the presentation, the information processing device of the present invention can support selection of medicine performed by the medical professional. For example, when an antihypertensive agent is prescribed, prescription starts with a single agent in a small amount, and, when an antihypertensive effect is barely obtained, a change to another antihypertensive agent can be supported. Further, when the antihypertensive effect is insufficient, the amount is increased or another type of an antihypertensive agent is also given in a small amount. In this case, it is known that a combination of different types of other antihypertensive agents in small amounts results in a better antihypertensive effect than doubling the amount of the antihypertensive agent. It is also known that a combination of different classes (for example, different types) of antihypertensive agents has a great antihypertensive effect and is useful for achieving an antihypertensive target. Therefore, it is possible to provide an information processing device capable of supporting prescription of a medicine appropriate for a user.

The information processing device according to one aspect described above further includes a storage unit configured to store the information about the medicine, the effect generation time period, and the information about the living body in association with one another, and the presentation control unit presents a type and a quantity of a medicine expected to have an effect, based on information stored in the storage unit.

In the configuration described above, since the information about the medicine, the effect generation time period, and the biological information are stored in association with one another, the type and quantity of a medicine that is effective for each living body can be presented, for example. It is also possible to present which combination among a plurality of combinations of medicines that are effective for each living body. Therefore, it is possible to provide an information processing device capable of supporting prescription of a medicine appropriate for a user.

In the information processing device according to one aspect described above, the information about the medicine includes a type of a medicine, an effect of a medicine, a use of a medicine, and a side effect of a medicine.

In the configuration described above, the information about the medicine can provide a medicine that is effective on a user and has few side effects to each user by including a type of a medicine, an effect of a medicine, a use of a medicine, and a side effect of a medicine. Therefore, it is possible to provide an information processing device capable of supporting prescription of a medicine that is effective on the user and has few side effects.

In the information processing device according to one aspect described above, the information about the living body includes a blood pressure value or a pulse.

In the configuration described above, for example, an effect and a side effect of a medicine can be determined by measuring the blood pressure value or the pulse. Note that the blood pressure value or the pulse is measured here, but any biological information may be used as long as a state (for example, numerical value information) different from that at a normal time is indicated by taking a medicine.

A biological information measuring device according to one aspect described above includes a measurement control unit configured to perform processing for measuring information about a living body, based on time information about when a medicine is to be taken based on information about the medicine, and an effect generation time period in which an effect of the medicine is estimated to be generated, and a transmission control unit configured to transmit the information about the living body.

In the configuration described above, the biological information measuring device can perform the processing for measuring biological information from the time information about when the medicine is to be taken and the effect generation time period, and transmit the biological information. In this way, a user wears, for example, the biological information measuring device, and biological information measured by the biological information measuring device is transmitted, and thus the biological information can be analyzed by a device other than the biological information measuring device.

The information processing device according to one aspect described above further includes a transmission control unit configured to transmit time information including the effect generation time period to a biological information measuring device configured to measure the information about the living body, and the acquisition control unit receives, from the biological information measuring device, the information about the living body measured by the biological information measuring device in response to the transmission of the time information.

In the configuration described above, the biological information measuring device can acquire information including the effect generation time period from the information processing device. Furthermore, the biological information measuring device can transmit the biological information to the information processing device, based on the effect generation time period. Thus, the information processing device can acquire biological information measured in the time period in which the effect of the medicine is generated.

In the information processing device according to one aspect described above, the estimation control unit acquires information about a living body at a plurality of times associated with a period assumed to be a mealtime, and estimates the time information, based on a change in the pieces of information about the living body.

In the configuration described above, the period for taking a medicine is set in accordance with the mealtime, and thus the plurality of times associated with the period assumed to be the mealtime is a time at which the medicine generates an effect or a time after a lapse of a certain period of time since the time at which the medicine generates the effect. The time at which the medicine has been taken can be estimated by observing the change in biological information at the plurality of times.

The information processing device according to one aspect described above further includes a display control unit configured to display the one or more medicines.

In the configurations described above, one or more medicines can be displayed from a plurality of medicines including a medicine expected to have an effect. Thus, for example, it is easier for a medical professional to select a medicine by viewing the displayed medicine.

In the information processing device according to one aspect described above, the time information is input by a user.

In the configuration described above, the user can directly input a time at which a medicine is to be taken.

In the biological information measuring device according to one aspect described above, the acquisition unit acquires the information about the medicine via a bar code.

In the configuration described above, the information about the medicine can be acquired by the user scanning the bar code described in the description of the medicine provided with the medicine. For example, the bar code can be easily scanned by a mobile information terminal.

In the biological information measuring device according to one aspect described above, a time at which the medicine is to be taken is presented based on the information about the medicine.

In the configuration described above, the time at which the medicine is to be taken is clear from a use of the medicine and the like included in the information about the medicine, and thus the time at which the medicine is to be taken can be presented to a user. Thus, the user is more likely to reliably take the medicine without forgetting to take the medicine.

In the biological information measuring device and the information processing device according to one aspect described above, information is input by touching a button formed of hardware or software.

In the configuration described above, the biological information measuring device and the information processing device include a button of hardware that is physically present or a button formed of software, and can receive a determination that the information may be provided to a third-party provision by a user touching the button.

In the biological information measuring device according to one aspect described above, a short-range wireless communication system is used for communication between the biological information measuring device and the information processing device.

In the configuration described above, the short-range wireless communication system is used for the communication between the biological information measuring device and the information processing device, and thus, for example, Bluetooth (registered trademark) is used. In addition, there are communication methods such as TransferJet (registered trademark), ZigBee (registered trademark), and IrDA (registered trademark), and these may be used.

The biological information measuring device according to one aspect described above is formed of the mobile information terminal and a biological information measuring instrument, and the mobile information terminal includes the transmission control unit, and the biological information measuring instrument includes a sensor configured to measure the information about the living body and a transmission control unit configured to transmit the information about the living body to the mobile information terminal.

In the configuration described above, the mobile information terminal is, for example, a smart phone, and the biological information measuring instrument is, for example, a blood pressure monitor having a transmission function. The mobile information terminal includes the main control unit in such a manner, and thus the biological information measuring instrument may only include a function of measuring biological information and a function of transmitting the biological information to the mobile information terminal. As a result, the mobile information terminal performs main control, and thus an existing biological information measuring instrument (for example, existing blood pressure monitor) can be used. Further, the short-range wireless communication system described above may be used for communication between the mobile information terminal and the biological information measuring instrument.

According to the present invention, it is possible to provide an information processing device, a biological information measuring device, a method, and a non-transitory recording medium in which a program is stored, capable of automatically acquiring biological information in a time period in which an effect of a medicine is generated.

### BRIEF DESCRIPTION OF DRAWINGS

FIG. 1 is a diagram schematically illustrating an example of an application scene of a system including an information processing device and a blood pressure measurement device according to an embodiment.

FIG. 2 is a diagram schematically illustrating an example of a hardware configuration of the blood pressure measurement device according to the embodiment.

FIG. 3 is a diagram illustrating an example of an appearance of the blood pressure measurement device in FIG. 2.

FIG. 4 is a diagram schematically illustrating an example of a hardware configuration of the information processing device according to the embodiment.

FIG. 5 is a diagram schematically illustrating an example of a software configuration of the blood pressure measurement device according to the embodiment.

FIG. 6 is a diagram schematically illustrating an example of a software configuration of the information processing device according to the embodiment.

FIG. 7 is a diagram illustrating an example of a processing procedure of the blood pressure measurement device and the information processing device according to the embodiment.

FIG. 8 is a diagram illustrating an example of a processing procedure of the blood pressure measurement device and the information processing device according to the embodiment.

FIG. 9 is a diagram illustrating an example of a processing procedure of the blood pressure measurement device and the information processing device according to the embodiment.

FIG. 10 is a diagram illustrating an example of the processing procedure in step S708 in FIG. 7 or step S805 in FIG. 8.

FIG. 11 is a diagram schematically illustrating an example of the software configuration of the information processing device according to a modified example.

FIG. 12 is a diagram schematically illustrating another example of the software configuration of the information processing device according to a modified example.

FIG. 13 is a diagram schematically illustrating an example of the hardware configuration of the blood pressure measurement device according to a modified example.

### DESCRIPTION OF EMBODIMENTS

An embodiment (hereinafter, also referred to as “the present embodiment”) according to an aspect of the present invention will be described below with reference to the drawings. Note that, in the following embodiment, portions provided with the same number are regarded as portions operating similarly, and thus redundant description thereof will be omitted.

### Application Example

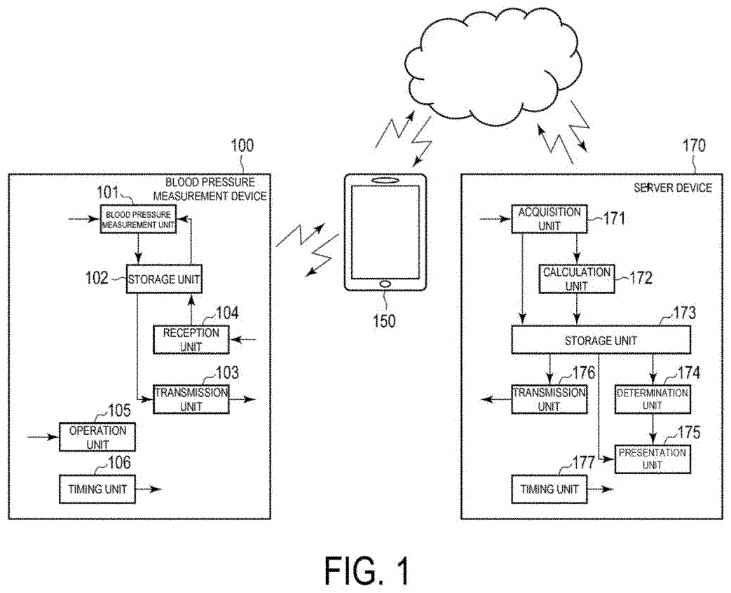

First, an example of a scene to which the present invention is applied will be described by using FIG. 1. FIG. 1 schematically illustrates an example of an application scene of a blood pressure measurement device 100 and a server device (hereinafter, also simply referred to as a “server”) 170 according to the present embodiment. In the blood pressure measurement device 100 according to the present embodiment, a reception unit 104 receives time information about when a medicine (for example, an antihypertensive agent) is to be taken and an effect generation time period, and a blood pressure measurement unit 101 measures blood pressure (an example of “information about a living body” of the present invention) at the time at which the medicine is to be taken and in the effect generation time period. The time information, the effect generation time period, and a measured blood pressure value are stored in a storage unit 102, and a transmission unit 103 transmits the blood pressure value in association with a measurement time. A time acquired by a timing unit 106 is used as the measurement time. An operation of the blood pressure measurement device 100 is performed via an operation unit 105. Then, a mobile information terminal 150 serves as a medium for communication between the blood pressure measurement device 100 and the server device 170. The mobile information terminal 150 communicates with the server device 170 via, for example, a network, and communicates with the blood pressure measurement device 100 through, for example, near-field wireless communication. Of course, in a case where it is possible, the blood pressure measurement device 100 may be set so as to directly communicate with the server device 170. Note that the blood pressure measurement device 100 and the mobile information terminal 150 correspond to a “biological information measuring device” of the present invention. Further, when the blood pressure measurement device 100 is set so as to be directly connected to the server device 170 via the network, the mobile information terminal 150 is unnecessary, and the blood pressure measurement device 100 corresponds to the “biological information measuring device” of the present invention.

In the server device 170 according to the present embodiment, an acquisition unit 171 acquires information transmitted from the mobile information terminal 150 and/or information on the network. The acquisition unit 171 acquires information about a medicine and time information about when the medicine is to be taken from a certain node on the network, and a calculation unit 172 calculates an effect generation time period in which an effect of the medicine is estimated to be generated, based on the pieces of information. Furthermore, the acquisition unit 171 acquires time series data (hereinafter, also simply referred to as “blood pressure measurement data”) about a blood pressure value from the blood pressure measurement device 100 via the mobile information terminal 150. A storage unit 173 stores the information about the medicine, the data, and the information about the effect generation time period, and a transmission unit 176 transmits the information about the medicine, the time information about when the medicine is to be taken, and the information about the effect generation time period to the blood pressure measurement device 100 via the mobile information terminal 150. Then, a determination unit 174 determines whether a medicine is effective on a living body, based on the blood pressure measurement data, and a presentation unit 175 displays the medicine information along with the determination result. Further, a time acquired by a timing unit 177 is used as the time. Note that the server device 170 corresponds to the “information processing device” of the present invention. Note that, instead of the server device 170, processing similar to the processing of the server device 170 may be performed by an application installed on the mobile information terminal 150. In this case, the server device 170 may be unnecessary. Furthermore, the blood pressure measurement device 100 may include a function of performing a function similar to that of the server device 170. In this case, the mobile information terminal 150 and the server device 170 may be unnecessary.

Any blood pressure measurement method may be used as long as the blood pressure measurement unit 101 performs a blood pressure measurement. The blood pressure measurement unit 101 measures blood pressure by, for example, an oscillometric method. Further, the blood pressure measurement device 100 may measure a pulse without performing a blood pressure measurement (or in addition to a blood pressure measurement). In other words, the blood pressure measurement device 100 in the present embodiment may measure any biological information that can confirm an effect of a medicine. Further, the blood pressure measurement device 100 may be a separate device other than the blood pressure measurement unit 101, the transmission unit 103, and the reception unit 104. In other words, the storage unit 102, the operation unit 105, and the timing unit 106 may be included in a separate device, for example, a mobile information terminal such as a smart phone, a mobile phone, or a mobile personal computer. In this case, the blood pressure measurement unit 101 is a blood pressure measuring instrument, measures blood pressure of a living body, and transmits measured blood pressure measurement data to the separate device described above. For example, a short-range wireless communication system (for example, Bluetooth) is used for communication between the blood pressure meter and the separate device. Further, the communication between the blood pressure meter and the separate device may also be achieved in a wired manner instead of a wireless manner (for example, one-to-one communication using a Universal Serial Bus (USB) cable or the like).

As described above, in the present embodiment, in the server device 170, the acquisition unit 171 can acquire information about a medicine scheduled to be taken and time information about when the medicine is to be taken from the network, and the calculation unit 172 can calculate an effect generation time period, based on the pieces of information. The transmission unit 176 transmits the effect generation time period, the information about the medicine, and the time information about when the medicine is to be taken to the blood pressure measurement device 100 via the mobile information terminal 150, and stores the effect generation time period, the information about the medicine, and the time information about when the medicine is to be taken in the storage unit 102 of the blood pressure measurement device 100. The blood pressure measurement unit 101 measures blood pressure, based on the effect generation time period and the dosing time stored in the storage unit 102. The measured blood pressure measurement data and/or blood pressure value information (also referred to simply as “blood pressure information”) is stored in the storage unit 102. Therefore, the blood pressure measurement device 100 can automatically acquire the blood pressure information in the effect generation time period.

The transmission unit 103 transmits the measured blood pressure information to the server device 170 via the mobile information terminal 150. The blood pressure information is at least two types of blood pressure information about the effect generation time period at a normal time and after the medicine is taken. The two types of the blood pressure information are acquired by the acquisition unit 171 of the server device 170 and stored in the storage unit 173. The determination unit 174 determines whether the medicine (for example, the antihypertensive agent) is effective on a user, based on the two types of the blood pressure information. Furthermore, when the determination unit 174 determines that there is no effect, the presentation unit 175 presents one or more medicines from a plurality of medicines expected to have an effect. Further, when the determination unit 174 determines that there is an effect, the presentation unit 175 may present that the medicine is effective. Therefore, the server device 170 can support prescription of a medicine appropriate for the user.

### Configuration Example

### Hardware Configuration

### Blood Pressure Measurement Device

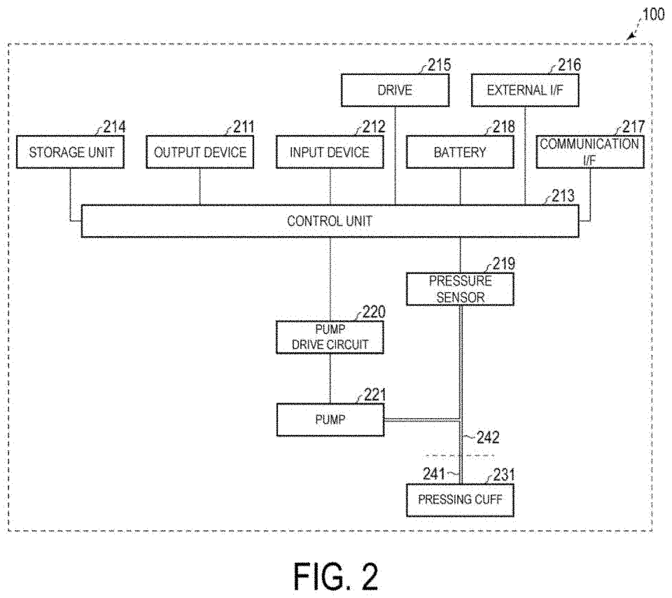

Next, an example of a hardware configuration of the blood pressure measurement device 100 according to the present embodiment will be described by using FIG. 2.

As illustrated in FIG. 2, the blood pressure measurement device 100 according to the present embodiment includes a computer to which an output device 211, an input device 212, a control unit 213, a storage unit 214, a drive 215, an external interface 216, a communication interface 217, and the battery 218 are electrically connected. The blood pressure measurement device 100 further includes a pressure sensor 219, a pump drive circuit 220, a pump 221, and a pressing cuff 231. Note that, in FIG. 2, the communication interface and the external interface are described as “communication I/F” and “external I/F”, respectively.

The control unit 213 includes a central processing unit (CPU), a random access memory (RAM), a read only memory (ROM), and the like, and controls each component according to information processing. The storage unit 214 is, for example, an auxiliary storage device such as a hard disk drive and a solid state drive, and stores a blood pressure measurement time setting program and a blood pressure measurement program that are executed by the control unit 213, and/or blood pressure measurement data being data about blood pressure measured by the blood pressure measurement device.

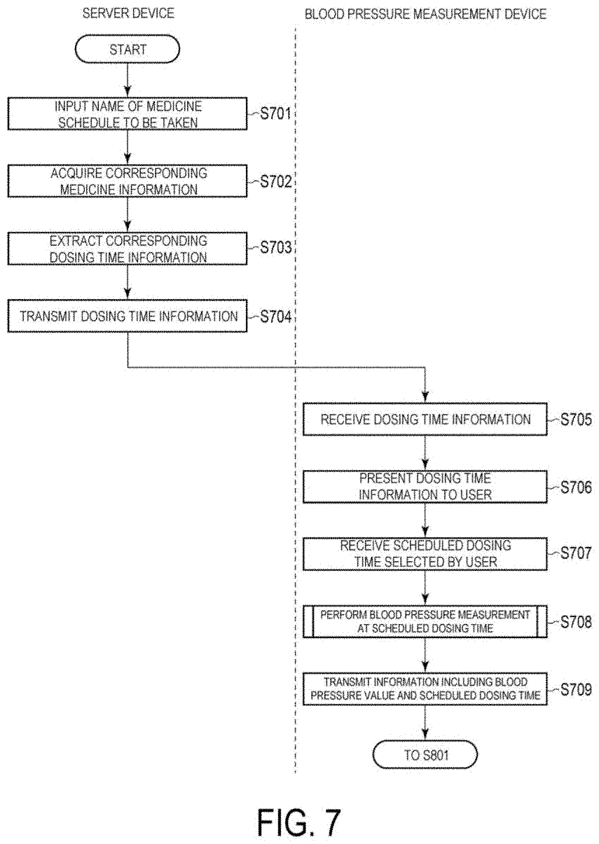

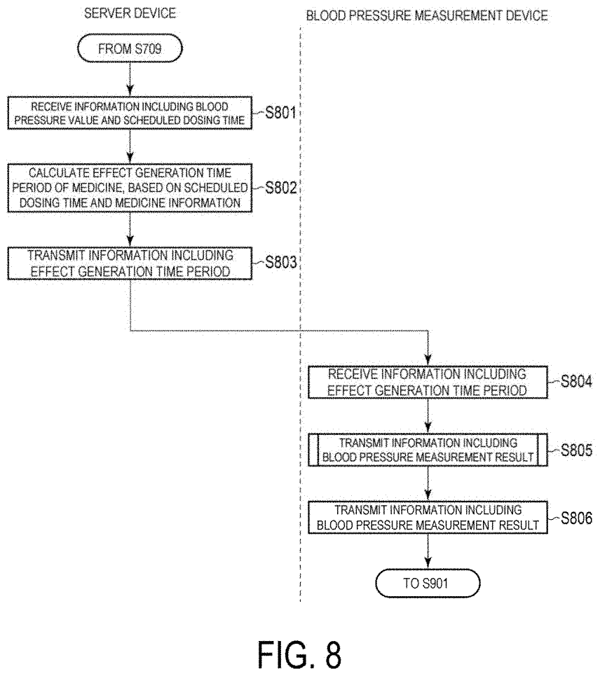

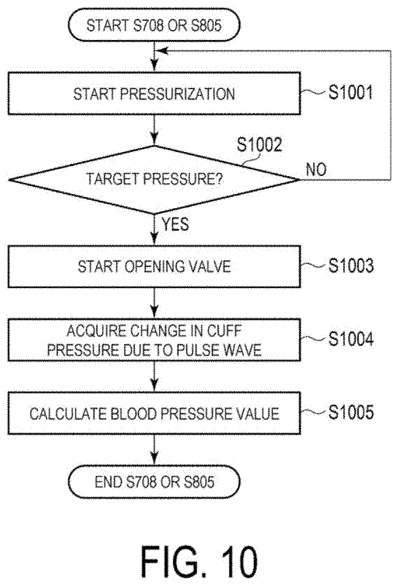

The blood pressure measurement time setting program is a program for causing execution of processing of setting a time at which a blood pressure measurement is performed, based on dosing time information and furthermore, setting a time at which the blood pressure measurement is performed, based on the effect generation time period (FIGS. 7 and 8). Further, the blood pressure measurement program is a program for causing execution of processing (FIG. 10) of measuring blood pressure of a user wearing a cuff by using the pressing cuff 231. Furthermore, the blood pressure measurement data are time series data about blood pressure acquired by executing the blood pressure measurement program. Details will be described below.

The communication interface 217 is, for example, a short-range wireless communication system (for example, Bluetooth) module, a wireless local area network (LAN) (for example, Wi-Fi (registered trademark)) module, and the like, and is an interface for performing wireless communication via a network. The communication interface 217 is an interface for connecting the blood pressure measurement device 100 to the mobile information terminal 150. The communication interface 217 is controlled by the control unit 213. The communication interface 217 passes information from the mobile information terminal 150 to the control unit 213. The communication may be wireless or wired. Note that the communication interface 217 may be able to directly transmit information to the server device 170 via the network. The network may be another type of a network such as an intra-hospital LAN or one-to-one communication using a Universal Serial Bus (USB) cable or the like. The communication interface 217 may include a micro USB connector.

Further, the communication interface 217 may be connected to the server device 170 via a separate device, for example, the mobile information terminal 150 such as a smart phone, a mobile phone, and a mobile personal computer, and the mobile information terminal 150 may perform all processing other than a blood pressure measurement (processing other than S708 in FIG. 7 and S805 and S806 in FIG. 8).

The input device 212 is a device for performing input of, for example, a mouse, a keyboard, or the like. The output device 211 is a device for performing output of, for example, a display, a speaker, or the like. The external interface 216 is a USB port or the like, and is an interface for establishing connection to an external device, such as the pressure sensor 219 and/or the pump drive circuit 220, for example.

The storage unit 214 is a medium that accumulates information about a recorded program and the like by electrical, magnetic, optical, mechanical, or chemical action such that the information about the program and the like can be read by a computer, another device, a machine, and the like. The blood pressure measurement device 100 may acquire the blood pressure measurement time setting program, the blood pressure measurement program, and/or the blood pressure measurement data from the storage unit 214.

The drive 215 is, for example, a Compact Disk (CD) drive, a Digital Versatile Disk (DVD) drive, or the like, and is a device for reading a program stored in a storage medium. A type of the drive 215 may be appropriately selected according to a type of the storage medium. The blood pressure measurement time setting program, the blood pressure measurement program, and/or the blood pressure measurement data described above may be stored in the storage medium. Here, as an example of the storage medium, a disk type storage medium such as a CD and a DVD is illustrated. However, a type of the storage medium is not limited to a disk type, and may be other than a disk type. Examples of a storage medium other than a disk type may include a semiconductor memory such as flash memory, for example.

The battery 218 is, for example, a rechargeable secondary battery. The battery 218 supplies power to each element mounted on a body of the blood pressure measurement device 100. The battery 218 supplies power to, for example, the output device 211, the input device 212, the control unit 213, the storage unit 214, the drive 215, the external interface 216, the communication interface 217, the pressure sensor 219, the pump drive circuit 220, and the pump 221.

The pressure sensor 219 is, for example, a piezoresistive pressure sensor. The pressure sensor 219 detects pressure in the pressing cuff 231 via a flexible tube 241 and a first flow path formation member 242 that constitute a first flow path. The pressure sensor 219 outputs pressure data (for example, time series data about a pressure value) to the control unit 213.

The pump drive circuit 220 drives or brakes the pump 221 (that is, turns on or off the pump 221), based on a control signal from the control unit 213.

The pump drive circuit 220 drives the pump 221 that injects fluid into the pressing cuff 231 when it is determined that fluid is to be injected.

The pump 221 is, for example, a piezoelectric pump. The pump 221 is connected to the pressing cuff 231 via the first flow path such that fluid can flow. The pump 221 can supply fluid (for example, air) to the pressing cuff 231 through the first flow path. Note that an exhaust valve (not illustrated) whose opening and closing is controlled as the pump 221 is turned on or off is mounted on the pump 221. In other words, the exhaust valve closes when the pump 221 is turned on, and encloses air within the pressing cuff 231. On the other hand, the exhaust valve opens when the pump 221 is turned off, and discharges air in the pressing cuff 231 into the atmosphere through the first flow path. Note that the exhaust valve functions as a check valve, and prevents the discharged air from flowing in reverse.

Further, unlike this, the control unit 213 may control on or off of the pump 221 and control opening and closing of the exhaust valve separately.

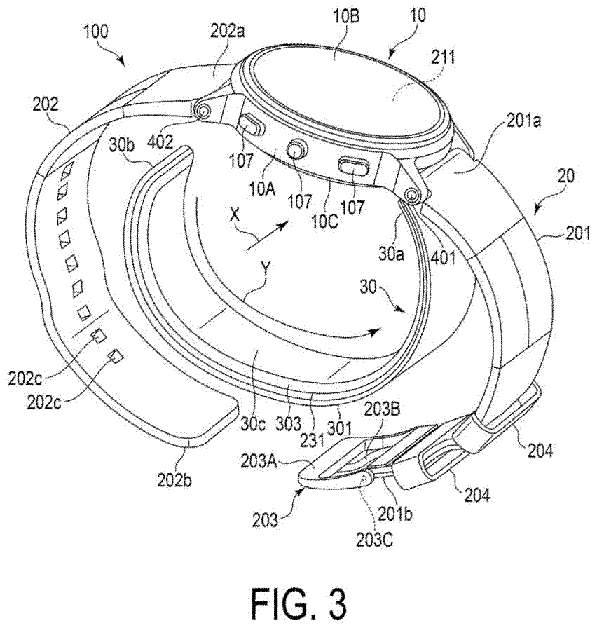

The pressing cuff 231 will be described below with reference to FIG. 3.

Further, the amount of fluid injected to the pressing cuff 231 by the pump 221 may be fixed. The pump 221 supplies a fixed flow rate at 10 sccm (standard cubic centimeter per minute), for example. sccm is standardized at atmospheric pressure and temperature (for example, standardized at 1 atmospheric pressure and 0 degree Celsius). In this case, when an elapsed time since the injection starts is clear, the amount of fluid injected into the pressing cuff 231 can be calculated. In the present embodiment, the amount of fluid injected into the pressing cuff 231 can be calculated by measuring an elapsed time since the control unit 213 turns on the pump drive circuit 220.

Further, a data table representing a relationship between a drive voltage of the pump drive circuit 220 and a flow rate may be previously stored in the storage unit 214, and the control unit 213 may acquire, from the drive voltage of the pump drive circuit 220, the flow rate by replacing a voltage value with the flow rate with reference to the data table.

Note that, with regard to a specific hardware configuration of the blood pressure measurement device 100, a component can be omitted, replaced, and added as appropriate according to the embodiment. For example, the control unit 213 may include a plurality of processors. The blood pressure measurement device 100 may be constituted by a plurality of information processing devices. Further, in addition to an information processing device designed to be dedicated to a service provided, a general-purpose desktop personal computer (PC), a tablet PC, and the like may be used for the blood pressure measurement device 100.

### Structure of Blood Pressure Measurement Device

Next, an example of a structure of the blood pressure measurement device 100 will be described with reference to FIG. 3.

FIG. 3 is a diagram illustrating an example of an appearance of the blood pressure measurement device according to the invention.

The blood pressure measurement device 100 is, for example, a wrist watch-type wearable device. The blood pressure measurement device 100 includes a body 10, a belt 20, and a cuff structure 30.

The body 10 is configured to allow mounting of a plurality of elements, such as an element of a control system of the blood pressure measurement device 100.

The body 10 includes a case 10A, a glass 10B, and a back lid 10C.

The case 10A has, for example, a substantially short cylindrical shape. The case 10A includes, at two locations on each side surface thereof, a pair of protruding lugs for attaching the belt 20.

The glass 10B is attached to an upper portion of the case 10A. The glass 10B has, for example, a circular shape.

The back lid 10C is removably attached to a lower portion of the case 10A so as to face the glass 10B.

The body 10 includes the output device 211 and an operation unit 107 mounted thereon.

The output device 211 displays various types of information. The output device 211 is provided in the body 10 in a position that can be visually recognized by a subject through the glass 10B. The output device 211 is constituted by a liquid crystal display (LCD), for example. The output device 211 may be an organic electro luminescence (EL) display. The output device 211 may include a function of displaying various types of information, and is not limited thereto. The output device 211 may include a light emitting diode (LED).

The operation unit 107 is included in the input device 212, and is an element for inputting various types of instructions to the blood pressure measurement device 100. The operation unit 107 is provided on the side surface of the body 10. The operation unit 107 includes one or more push type switches, for example. The operation unit 107 may be, for example, a pressure sensitive type (resistance type) or a proximity type (capacitance type) touch panel type switch. The operation unit 107 may include a function of inputting various types of instructions to the blood pressure measurement device 100, and is not limited thereto.

Here, an example of the switch included in the operation unit 107 will be described.

The operation unit 107 includes a measurement switch for instructing a start or a stop of a blood pressure measurement. Further, the operation unit 107 may include a home switch for returning a display screen of the output device 211 to a predetermined home screen, and a recording calling switch for causing the output device 211 to display a measurement record such as past blood pressure and the amount of activity.

Note that a plurality of elements other than the output device 211 and the operation unit 107 are mounted on the body 10. The plurality of elements mounted on the body 10 will be described below.

The belt 20 is configured to be able to wrap a target measurement site (for example, a left wrist, a right wrist) of a subject (user). It is assumed that a width direction of the belt 20 is an X direction. On the other hand, it is assumed that a direction in which the belt 20 wraps the target measurement site is a Y direction.

The belt 20 includes a first belt portion 201, a second belt portion 202, a buckle 203, and a belt holding portion 204.

The first belt portion 201 is a band-like belt portion extending from the body 10 to one side (right side in FIG. 3) in one direction. A root portion 201a of the first belt portion 201 near the body 10 is freely rotatably attached to the pair of lugs of the body 10 via a connecting rod 401.

The second belt portion 202 is a band-like belt portion extending from the body 10 to the other side (left side in FIG. 3) in one direction. A root portion 202a of the second belt portion 202 near the body 10 is freely rotatably attached to the pair of lugs of the body 10 via a connecting rod 402. A plurality of small holes 202c are formed through the second belt portion 202 in the thickness direction between the root portion 202a of the second belt portion 202 and a tip portion 202b farther from the body 10.

The buckle 203 is configured to be able to fasten the first belt portion 201 and the second belt portion 202. The buckle 203 is attached to a tip portion 201b of the first belt portion 201 farther from the body 10. The buckle 203 includes a frame body 203A, a prong 203B, and a connecting rod 203C.

The frame body 203A and the prong 203B are freely rotatably attached to the tip portion 201b of the first belt portion 201 via the connecting rod 203C. The frame body 203A and the prong 203B are formed of a metal material, for example. The frame body 203A and the prong 203B may be formed of a plastic material. When the first belt portion 201 and the second belt portion 202 are fastened, the tip portion 202b of the second belt portion 202 is passed through the frame body 203A. The prong 203B is inserted through any one of the plurality of small holes 202c of the second belt portion 202.

The belt holding portion 204 is attached between the root portion 201a and the tip portion 201b of the first belt portion 201. When the first belt portion 201 and the second belt portion 202 are fastened, the tip portion 202b of the second belt portion 202 is passed through the belt holding portion 204.

### Structure of Cuff

A configuration of the cuff structure 30 will be described.

The cuff structure 30 is configured to be able to compress a target measurement site during a blood pressure measurement.

The cuff structure 30 is a band-like cuff structure extending along the Y direction. The cuff structure 30 faces an inner circumferential surface of the belt 20. A first end 30a of the cuff structure 30 is attached to the body 10. A second end 30b of the cuff structure 30 is a free end. Thus, the cuff structure 30 is freely spaced from the inner circumferential surface of the belt 20.

The cuff structure 30 includes a curler 301, a pressing cuff 231, and a back plate 303.

The curler 301 is disposed at an outermost circumference of the cuff structure 30. In a natural state, the curler 301 is curved along the Y direction. The curler 301 is a resin plate having predetermined flexibility and hardness. The resin plate is made of polypropylene, for example.

The pressing cuff 231 is disposed along an inner circumferential surface of the curler 301. The pressing cuff 231 has a bag shape. The flexible tube 241 (FIG. 2) is attached to the pressing cuff 231. The flexible tube 241 is an element for supplying fluid from the body 10 side or discharging fluid from the pressing cuff 231. The fluid is, for example, air. When fluid is supplied to the pressing cuff 231, the pressing cuff 231 is inflated, and compresses the target measurement site.

Note that the pressing cuff 231 may include two fluid bags laminated in the thickness direction, for example. Each of the fluid bags is constituted of a stretchable polyurethane sheet, for example. When fluid is supplied to the pressing cuff 231, the fluid flows into each of the fluid bags. The inflation of each of the fluid bags inflates the pressing cuff 231.

The back plate 303 is disposed along an inner circumferential surface of the pressing cuff 231. The back plate 303 has a band shape. The back plate 303 is made of a resin, for example. The resin is polypropylene, for example. The back plate 303 functions as a reinforcing plate.

A plurality of grooves that extend in the X direction and have a V-shape or U-shape cross-section are spaced apart from each other in the Y direction and provided in parallel in an inner circumferential surface and an outer circumferential surface of the back plate 303. Since the back plate 303 is easily bent, the back plate 303 does not prevent the cuff structure 30 from being curved.

### Server Device

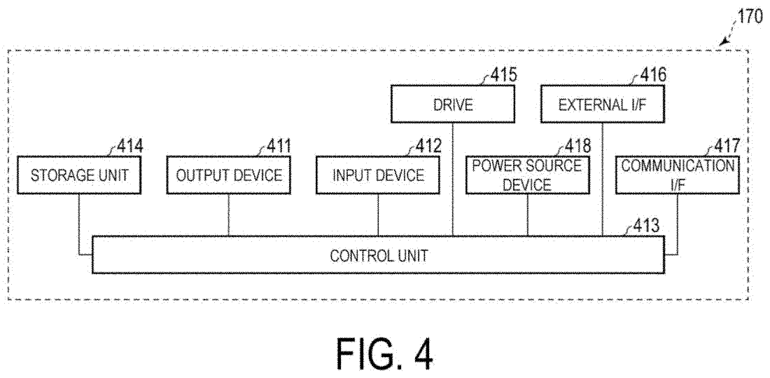

Next, an example of a hardware configuration of the server device 170 according to the present embodiment will be described by using FIG. 4.

As illustrated in FIG. 4, the server device 170 according to the present embodiment includes a computer to which an output device 411, an input device 412, a control unit 413, a storage unit 414, a drive 415, an external interface 416, a communication interface 417, and a power source device 418 are electrically connected. Note that, in FIG. 4, the communication interface and the external interface are described as “communication I/F” and “external I/F”, respectively.

The control unit 413 includes a central processing unit (CPU), a random access memory (RAM), a read only memory (ROM), and the like, and controls each component according to information processing. The storage unit 414 is, for example, an auxiliary storage device such as a hard disk drive and a solid state drive, and stores an effect generation time period-calculation program and an effect determination presentation program that are executed by the control unit 413, and/or data transmitted and/or received by the server device or transmission-reception data.

The effect generation time period-calculation program is a program for causing execution of processing (FIG. 7) of acquiring medicine information, based on a name of a medicine scheduled to be taken (for example, from a network), and extracting time information about when the medicine is to be taken from the medicine information. Furthermore, the program is a program for causing execution of processing (FIG. 8) of receiving dosing time information, based on an instruction from a user, and calculating an effect generation time period from the dosing time information and the medicine information.

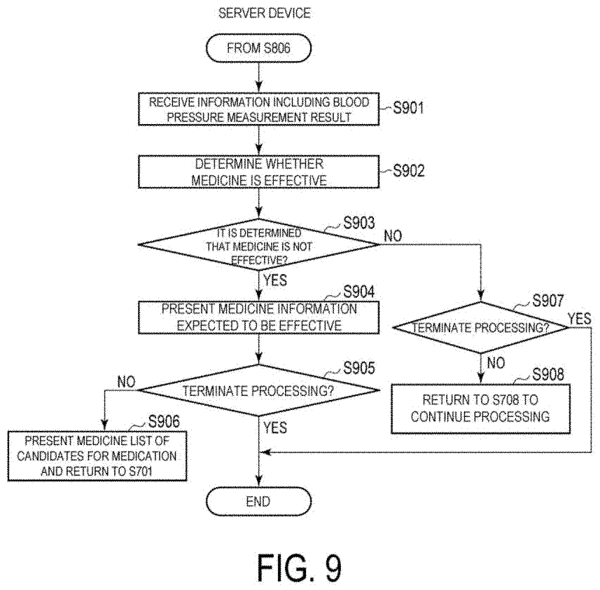

The effect determination presentation program is a program for causing execution of processing (FIG. 9) of determining whether a medicine is effective, based on blood pressure values and/or blood pressure measurement data at at least two different times (for example, at a normal time and in an effect generation time period). Furthermore, the program is a program for causing execution of processing (FIG. 9) for presenting the medicine information expected to be effective when it is determined that the medicine is not effective.

The communication interface 417 is, for example, a short-range wireless communication system (for example, Bluetooth) module, a wireless local area network (LAN) (for example, Wi-Fi) module, and the like, and is an interface for performing wireless communication via a network. The communication interface 417 is an interface for connecting the server device 170 to the mobile information terminal 150. The communication interface 417 is controlled by the control unit 413. The communication interface 417 passes information from the blood pressure measurement device 100 received via the network and the mobile information terminal 150 to the control unit 413. The communication via the network may be wireless or wired. Note that the communication interface 417 may be able to directly transmit information to the blood pressure measurement device 100 via the network. The network may be another type of a network such as an intra-hospital LAN or one-to-one communication using a Universal Serial Bus (USB) cable or the like. The communication interface 217 may include a micro USB connector.

The input device 412 is a device for performing input of, for example, a mouse, a keyboard, or the like. The output device 411 is a device for performing output of, for example, a display, a speaker, or the like. The external interface 416 is a USB port or the like, and is an interface for establishing connection to an external device.

The storage unit 414 is a medium that accumulates information about a recorded program and the like by electrical, magnetic, optical, mechanical, or chemical action such that the information about the program and the like can be read by a computer, another device, a machine, and the like. The server device 170 may acquire a third-party provision confirmation program and/or transmission-reception data from the storage unit 414.

The drive 415 is, for example, a Compact Disk (CD) drive, a Digital Versatile Disk (DVD) drive, or the like, and is a device for reading a program stored in a storage medium. A type of the drive 415 may be appropriately selected according to a type of the storage medium. The effect generation time period-calculation program, the effect determination presentation program, and/or the transmission-reception data may be stored in the storage medium. Here, as an example of the storage medium, a disk type storage medium such as a CD and a DVD is illustrated. However, a type of the storage medium is not limited to a disk type, and may be other than a disk type. Examples of a storage medium other than a disk type may include a semiconductor memory such as flash memory, for example.

The power source device 418 is, for example, a rechargeable secondary battery. The power source device 418 supplies power to each element mounted on a body of the server device 170. The power source device 418 supplies power to, for example, the output device 411, the input device 412, the control unit 413, the storage unit 414, the drive 415, the external interface 416, and the communication interface 417.

### Software Configuration

### Blood Pressure Measurement Device

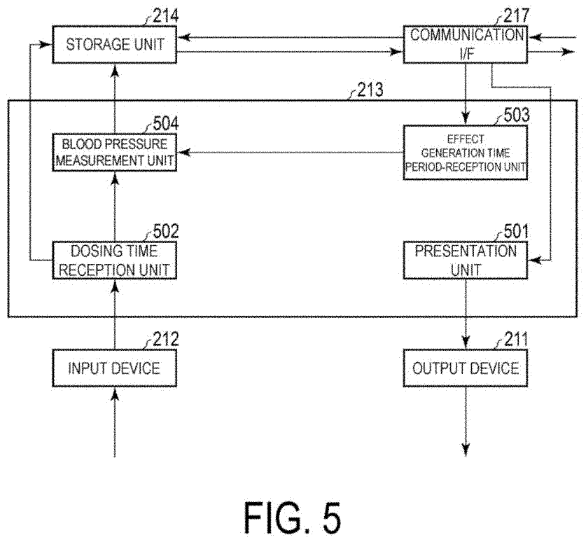

Next, an example of a software configuration of the blood pressure measurement device 100 according to the present embodiment will be described by using FIG. 5.

When a necessary program is executed, the control unit 213 of the blood pressure measurement device 100 develops, in the RAM, the blood pressure measurement time setting program and/or the blood pressure measurement program stored in the storage unit 214. Then, the control unit 213 interprets and executes the blood pressure measurement time setting program and/or the blood pressure measurement program developed in the RAM by the CPU, and controls each component. In this way, as illustrated in FIG. 5, the blood pressure measurement device 100 according to the present embodiment functions as a computer including a presentation unit 501, a dosing time reception unit 502, an effect generation time period-reception unit 503, and a blood pressure measurement unit 504.

The presentation unit 501 presents, to the user, the dosing time information received from the server device 170 via the communication interface 217. The user recognizes the time at which a medicine is to be taken from the dosing time information, and inputs a scheduled dosing time to the blood pressure measurement device 100 via the input device 212.

The dosing time reception unit 502 receives the dosing time input by the user via the input device 212 based on the dosing time information received from the server device 170. Furthermore, the dosing time reception unit 502 instructs the blood pressure measurement unit 504 to start a blood pressure measurement at a normal time of a living body, based on the received dosing time. For example, the dosing time reception unit 502 instructs the start of the blood pressure measurement at the dosing time. In addition, a blood pressure measurement start time instructed by the dosing time reception unit 502 may also be, for example, five minutes before the dosing time. In short, the dosing time reception unit 502 may perform a measurement on any date and time as long as blood pressure at the normal time at which the living body does not take a medicine can be measured. However, in order to determine the effect of taking a medicine, it is desirable that the blood pressure measurement start time is close to a dosing time (for example, before the dosing time). Further, the blood pressure measurement start time may be after a medicine is taken as long as the effect of the medicine is not achieved.

The effect generation time period-reception unit 503 receives the effect generation time period calculated by the server device 170 via the communication interface 217, and instructs the blood pressure measurement unit 504 to start the blood pressure measurement when the effect generation time period arrives.

The blood pressure measurement unit 504 starts the blood pressure measurement when receiving a start instruction of the blood pressure measurement from the dosing time reception unit 502 or the effect generation time period-reception unit 503. For example, the blood pressure measurement unit 504 starts the blood pressure measurement of the user by applying pressure to the cuff structure 30 by the pump drive circuit 220, and receives a pressure value of the cuff structure 30 being measured by the pressure sensor 219.

Further, any blood pressure measurement method may be used as long as the blood pressure measurement unit 504 performs a blood pressure measurement. The blood pressure measurement unit 504 measures blood pressure by, for example, an oscillometric method. A blood pressure value (systolic blood pressure value and diastolic blood pressure value) measured by the blood pressure measurement unit 504 is stored in the storage unit 214. As illustrated in FIG. 1, the blood pressure measurement device 100 may include the timing unit 106, and also acquire a measured time along with a blood pressure value. The storage unit 214 stores time series data about a blood pressure value, for example. Furthermore, the blood pressure measurement device 100 may include a device that acquires position information, and a position in which a blood pressure value is measured may also be stored in the storage unit 214 in association with a blood pressure value. The position information may be acquired by, for example, providing a GPS receiver in the blood pressure measurement device 100, or the position information acquired by the mobile information terminal 150 may be received on the assumption that the mobile information terminal 150 is always carried.

### Server Device

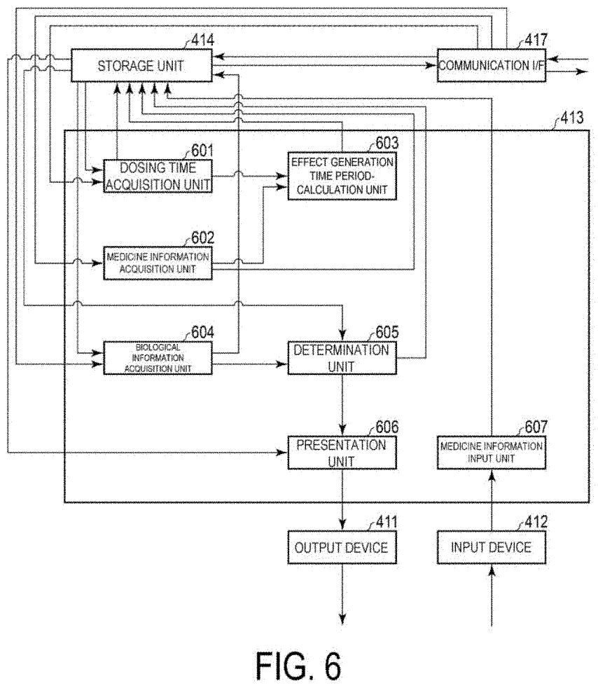

Next, an example of a software configuration of the server device 170 according to the present embodiment will be described by using FIG. 6.

When a necessary program is executed, the control unit 413 of the server device 170 develops, in the RAM, the effect generation time period-calculation program and/or the effect determination presentation program stored in the storage unit 414. Then, the control unit 413 interprets and executes the third-party provision confirmation program developed in the RAM by the CPU, and controls each component. In this way, as illustrated in FIG. 6, the server device 170 according to the present embodiment functions as a computer including a dosing time acquisition unit 601, a medicine information acquisition unit 602, an effect generation time period-calculation unit 603, a biological information acquisition unit 604, a determination unit 605, a presentation unit 606, and a medicine information input unit 607.

The dosing time acquisition unit 601 acquires a dosing time, which is time information about when a medicine is to be taken from the blood pressure measurement device 100 via the communication interface 417 and the mobile information terminal 150, and stores the dosing time in the storage unit 414. When, first, a medicine is set by the server device 170, the dosing time acquisition unit 601 acquires dosing time information from medicine information about the set medicine. The medicine information is acquired by the medicine information acquisition unit 602 and stored in the storage unit 414.

The medicine information acquisition unit 602 acquires the medicine information from the network via the communication interface 417 and stores the medicine information in the storage unit 414. The medicine information includes, for example, a type of a medicine, an effect of a medicine, a use of a medicine, and a side effect of a medicine.

The effect generation time period-calculation unit 603 acquires the dosing time from the dosing time acquisition unit 601 and the medicine information from the medicine information acquisition unit 602, and calculates a time period in which an effect of the medicine taken is generated from the pieces of information. Then, the effect generation time period-calculation unit 603 transmits the calculated information to the blood pressure measurement device 100.

The biological information acquisition unit 604 acquires biological information (for example, measurement time and blood pressure value) measured by the blood pressure measurement device 100 from the blood pressure measurement device 100 via the communication interface 417 and the mobile information terminal 150, and stores the biological information in the storage unit 414. Here, since the determination unit 605 in a next stage determines effectiveness of the medicine, the pieces of biological information at a normal time and in an effect generation time period, for example, are used.

The determination unit 605 determines whether the medicine taken by the user is effective on the user, based on the biological information acquired by the biological information acquisition unit 604 and the medicine information stored in the storage unit 414. For example, the determination unit 605 can determine whether the medicine taken by the user is effective on the user by comparing the biological information measured at the normal time (for example, the time at which the medicine is taken) stored in the storage unit 414 with the biological information measured in the effect generation time period. Furthermore, the determination unit 605 compares a value indicated by the biological information at the normal time with a value indicated by the biological information in the effect generation time period stored in the storage unit 414, takes a difference between the values, and determines whether the medicine taken is effective on the user from the sign of the difference and the absolute value of the difference.

When the determination unit 605 determines that the medicine is not effective, the presentation unit 606 displays, along with a combination, one or more medicines that are expected to have an effect from the medicine information stored in the storage unit 414. Note that it is desirable that a medicine determined not to be effective is also included in the combination. The reason for this is that, for example, in a case of an antihypertensive agent, a combination of different types of other antihypertensive agents in small amounts results in a better antihypertensive effect than doubling the amount of the antihypertensive agent.

Further, when the determination unit 605 determines that the medicine is effective, the presentation unit 606 may present information about the effective medicine. Further, when the determination unit 605 determines that the medicine is effective, the presentation unit 606 controls the server device 170 so as to receive medicine information via the output device 411 and the input device 412 in such a way to be determined by a medical professional. Furthermore, when the determination unit 605 determines that the medicine is not effective, the medical professional may determine whether taking of a new medicine starts via the input device 412, and the medicine information input unit 607 may receive new medicine information.

In this case, the presentation unit 606 presents a medicine list of candidates for medication, for example, and the medical professional determines a medicine to be selected.

The medicine information input unit 607 receives an input of a name of a medicine to be given to the user (patient), and stores the medicine information input by the input device 412 in the storage unit 414. Furthermore, the medicine information input unit 607 performs control such that the input device 412 can select a new medicine or a combination of new medicines from one or more medicines presented by the presentation unit 606, and stores information about the selected medicine in the storage unit 414. For example, the medical professional views a medicine presented by the presentation unit 606 with the output device 411, and inputs medicine information newly selected with the input device 412.

### Other

Each function of the blood pressure measurement device 100 and the server device 170 will be described in detail in an operation example described below. Note that, in the present embodiment, an example in which each function of the blood pressure measurement device 100 and the server device 170 is achieved by a general-purpose CPU is described. However, some or all of the above-described functions may be achieved by one or a plurality of dedicated processors. Further, with regard to the functional configuration of the blood pressure measurement device 100 and the server device 170, a function may be appropriately omitted, replaced, and added as appropriate according to the embodiment.

### Operation Example

### Blood Pressure Measurement Device and Server Device

Next, an operation example of the blood pressure measurement device 100 and the server device 170 will be described by using FIGS. 7, 8, and 9. FIGS. 7, 8, and 9 are a flowchart illustrating an example of a processing procedure of the blood pressure measurement device 100 and the server device 170. Note that the processing procedure described below is merely an example, and each processing may be changed as much as possible. Further, with regard to the processing procedure described below, a step can be omitted, replaced, and added as appropriate according to the embodiment.

### Activation

First, the server device 170 and the blood pressure measurement device 100 are activated, causing the activated server device 170 to execute the effect generation time period-calculation program and/or the effect determination presentation program, and causing the activated blood pressure measurement device 100 to execute the blood pressure measurement time setting program and/or the blood pressure measurement program. According to the following procedure, the control unit 413 of the server device 170 receives medicine information about a medicine scheduled to be taken, and acquires the medicine information and dosing time information from the network (steps S701 to S704), receives scheduled dosing time information from the blood pressure measurement device 100, calculates an effect generation time period of the medicine, and transmits information including the effect generation time period to the blood pressure measurement device 100 (steps S801 to S803). Then, the server device 170 receives blood pressure measurement result information of the blood pressure measurement device 100, determines effectiveness of the medicine, and determines subsequent processing according to the effectiveness (steps S901 to S908).

On the other hand, according to the following procedure, the control unit 213 of the blood pressure measurement device 100 receives the dosing time information from the server device 170, a user determines the scheduled dosing time, and the blood pressure measurement device 100 measures blood pressure at the scheduled time (steps S705 to S709). Then, the blood pressure measurement device 100 receives information including the effect generation time period from the server device 170, measures blood pressure in the effect generation time period, and transmits information including these blood pressure measurement results to the server device 170 (steps S804 to S806).

### Step S701

In step S701, the control unit 413 functions as the medicine information input unit 607, and inputs a name of a medicine scheduled to be taken by a user (patient) by the input device 412. The input is performed by, for example, a medical professional (doctor).

### Step S702

In step S702, the control unit 413 functions as the medicine information acquisition unit 602, and acquires medicine information from the network via the communication interface 417.

### Step S703

In step S703, the control unit 413 functions as the dosing time acquisition unit 601, and acquires information about a dosing time of the corresponding medicine from the medicine information acquired in step S702.

### Step S704

In step S704, the control unit 413 functions as the dosing time acquisition unit 601, and transmits the dosing time information extracted from the medicine information to the blood pressure measurement device 100 via the communication interface 417.

### Step S705

In step S705, the control unit 213 functions as the presentation unit 501, and receives the dosing time information included in the medicine information acquired by the server device 170 from the server device 170 via the communication interface 217.

### Step S706

In step S706, the control unit 213 functions as the presentation unit 501, and presents the dosing time information received in step S705 to the user via the output device 211.

### Step S707

In step S707, the control unit 213 functions as the dosing time reception unit 502, and receives information that is acquired by selecting the scheduled dosing time by the user and is input via the input device 212, based on the dosing time information presented in step S706.

### Step S708

In step S708, the control unit 213 functions as the blood pressure measurement unit 504, and measures blood pressure of the user at the scheduled dosing time received in step S707. Details of the blood pressure measurement performed by the blood pressure measurement device 100 will be described later with reference to FIG. 10.

### Step S709

In step S709, the control unit 213 functions as the blood pressure measurement unit 504, and transmits information including the blood pressure measurement value and the dosing time (which may be the scheduled dosing time) to the server device 170 via the communication interface 217.

### Step S801

In step S801, the control unit 413 functions as the dosing time acquisition unit 601 and the biological information acquisition unit 604. The dosing time acquisition unit 601 acquires the dosing time from the blood pressure measurement device 100 via the communication interface 417. The biological information acquisition unit 604 acquires biological information (here, the blood pressure value) measured at the dosing time from the blood pressure measurement device 100 via the communication interface 417.

### Step S802

In step S802, the control unit 413 functions as the effect generation time period-calculation unit 603, and calculates a time period in which the medicine taken generates an effect, based on the scheduled dosing time information received in step S801 and the medicine information acquired in step S702.

### Step S803

In step S803, the control unit 413 functions as the effect generation time period-calculation unit 603, and transmits information including the effect generation time period calculated in step S802 to the blood pressure measurement device 100 via the communication interface 417.

### Step S804

In step S804, the control unit 213 functions as the effect generation time period-reception unit 503, and receives the information including the effect generation time period transmitted from the server device 170.

### Step S805

In step S805, the control unit 213 functions as the blood pressure measurement unit 504, and performs a blood pressure measurement in the effect generation time period included in the information received by the effect generation time period-reception unit 503.

### Step S806

In step S806, the control unit 213 functions as the blood pressure measurement unit 504, and transmits information including the blood pressure measurement result to the server device 170 via the communication interface 217.

### Step S901

In step S901, the control unit 413 functions as the biological information acquisition unit 604, and receives the information including the blood pressure measurement result from the blood pressure measurement device 100 via the communication interface 417.

### Step S902

In step S902, the control unit 413 functions as the determination unit 605, and determines whether the medicine taken by the user is effective, based on two or more blood pressure measurement results.

### Step S903

In step S903, the control unit 413 functions as the determination unit 605, and instructs the presentation unit 606 to perform the following processing when determining that the medicine is not effective, based on the blood pressure measurement result. When the control unit 413 determines that the medicine is effective, the processing proceeds to step S907.

### Step S904

In step S904, the control unit 413 functions as the presentation unit 606, and presents, via the output device 411, medicine information expected to have an effect on the user from the medicine information stored in the storage unit 414.

### Step S905

In step S905, the control unit 413 functions as the presentation unit 606, and determines whether to terminate the processing here. When the processing is not terminated, the processing proceeds to step S906. The determination of whether to terminate the processing may be received by the server device 170 via the output device 411 and the input device 412 in such a way as to be determined by a medical professional. Note that the determination may be freely set by the medical professional.

### Step S906

In step S906, the control unit 413 functions as the presentation unit 606, and instructs the medicine information input unit 607 to receive an input of a new medicine. The presentation unit 606 presents a medicine list of candidates for medication, and the medicine information input unit 607 performs control such that the medical professional can select a new medicine or a combination of new medicines by using the input device 412. Then, the selected new medicine or the selected combination of medicines is stored in the storage unit 414, the processing returns to step S701, and the processing proceeds with, as an input, the medicine or the combination of medicines stored in the storage unit 414.

### Step S907

In step S907, the control unit 413 functions as the presentation unit 606, presents whether to terminate the processing via the output device 411 and the input device 412, and causes the medical professional to determine whether to terminate the processing.

### Step S908

In step S908, the control unit 413 functions as the presentation unit 606, the processing returns to step S708 in order to continue the processing, and a blood pressure measurement is performed.

Next, a measurement of a blood pressure value of the user by the blood pressure measurement device 100, when a blood pressure measurement is performed in step S708 in FIG. 7 and S805 in FIG. 8, will be described with reference to FIG. 10. The blood pressure measurement device 100 measures a blood pressure value of the user by an oscillometric method by the control unit 213 according to the flowchart in FIG. 10, for example.

### Activation

In step S1001 in FIG. 10, the blood pressure measurement device 100 executes the blood pressure measurement program. The control unit 213 of the blood pressure measurement device 100 measures a blood pressure value of the user according to the following processing procedure. Although a blood pressure measurement by an oscillometric method is described here, other methods may be used as long as a blood pressure value of the user can be measured.

### Step S1001

In step S1001, the control unit 213 functions as the blood pressure measurement unit 504, and the blood pressure measurement unit 504 passes an instruction for pressurization via the pump drive circuit 220, gives an instruction to pressurize the cuff structure 30 (that is, to inject fluid into the cuff structure 30) by using the pump 221, and pressurization starts.

In terms of hardware, at the start of the blood pressure measurement, the control unit 213 initializes a processing memory region of the RAM, and outputs a control signal to the pump drive circuit 220. The pump drive circuit 220 opens the exhaust valve of the pump 221, based on the control signal, and discharges air in the pressing cuff 231 of the cuff. Next, the control unit 213 performs control so as to adjust the pressure sensor 219 at 0 mmHg. Then, the pump drive circuit 220 closes the exhaust valve of the pump 221. Subsequently, the pump drive circuit 220 drives the pump 221, and performs control so as to inject fluid into the cuff structure 30. In this way, the cuff structure 30 is pressurized at the same pressure and is inflated.

### Step S1002

In step S1002, the control unit 213 functions as the blood pressure measurement unit 504, detects pressure of the cuff structure 30 by the pressure sensor 219, and determines whether a pressure value being a preset target is reached. When the pressure value being the target is not reached, the processing returns to step S1001. When the pressure value being the target is reached, the processing proceeds to a next step. Here, the pressure value being the target is a pressure value (for example, systolic blood pressure value+30 mmHg) that is sufficiently higher than a systolic blood pressure value of the user, and is previously stored in the storage unit 214, or is determined by the control unit 213 estimating a systolic blood pressure value by a predetermined calculation formula during the pressurization of the cuff structure 30.

### Step S1003

In step S1003, the control unit 213 functions as the blood pressure measurement unit 504, and, when the pressure of the cuff structure 30 reaches the pressure value being the preset target due to pressurization, the control unit 213 stops the pump 221 via the pump drive circuit 220, and then performs control so as to gradually open the exhaust valve of the pump 221. In this way, the pressing cuff 231 contracts and is also gradually depressurized.

### Step S1004

In step S1004, the control unit 213 functions as the blood pressure measurement unit 504, and, in the depressurizing process starting from step S1003, the pressure sensor 219 detects a pressure value of the cuff structure 30, and outputs a cuff pressure signal. Further, for a depressurization rate, a target depressurization rate being a target is set during pressurization of the cuff structure 30, and the blood pressure measurement unit 504 controls a degree of opening of the exhaust valve of the pump 221 so as to achieve the target depressurization rate.

### Step S1005

In step S1005, the control unit 213 functions as the blood pressure measurement unit 504, and calculates a blood pressure value (systolic blood pressure value and diastolic blood pressure value), based on the cuff pressure signal via the oscillometric method using a known algorithm. When the blood pressure value is calculated, the control unit 213 performs control so as to store the calculated blood pressure value in the storage unit 214. When the measurement is completed, the control unit 213 opens the exhaust valve of the pump 221 via the pump drive circuit 220, and performs control so as to discharge air of the cuff structure 30. Note that the calculation of the blood pressure value is not limited being performed in the depressurizing process and may be performed in the pressurizing process.

### Action and Effect

As described above, in the present embodiment, the server device 170 can calculate the effect generation time period in which the effect of the medicine is estimated to be generated, based on the information about the medicine acquired in step S702 and the time information about when the medicine is to be taken received in step S801 (step S802). Then, the blood pressure measurement device 100 receives the information including the effect generation time period (step S804), and biological information (for example, blood pressure value and/or pulse) can be measured in the effect generation time period. Further, biological information is measured at a time at which the effect of the medicine is not generated, for example, at which the medicine is taken (step S708). In this way, by comparing the different pieces of biological information with or without the generation of the medicine effect, whether the medicine is effective on the user can be determined (step S902).

### Modified Example

While the embodiments of the present invention have been described in detail above, the foregoing description is merely illustrative of the present invention in all respects. Needless to say, various improvements and modifications can be made without departing from the scope of the present invention. For example, the following changes are possible. Note that, in the following, the same reference numerals are used for components similar to those of the above-described embodiment, and description of points similar to those of the above-described embodiment will be omitted as appropriate. The following modified examples can be combined as appropriate.

1

### Software Configuration

### Server Device

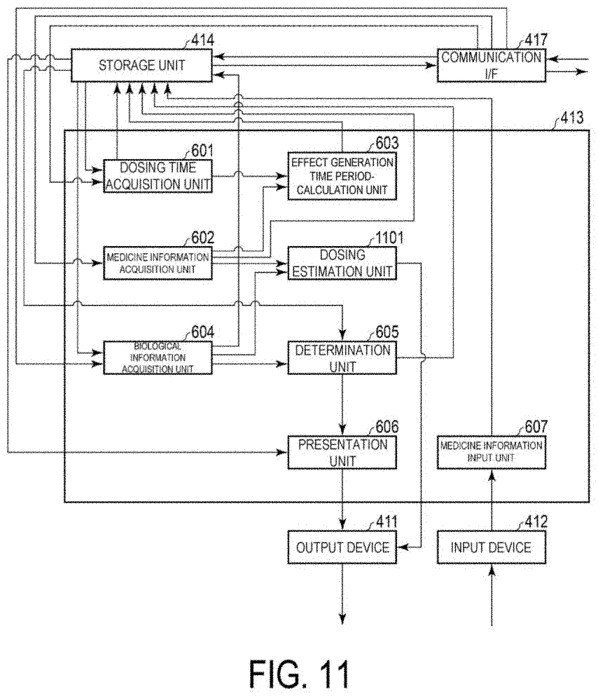

Next, an example of a modified example of the software configuration of the server device 170 according to the present embodiment will be described by using FIG. 11. A server device in the modified example is acquired by newly adding a dosing estimation unit 1101 to the server device 170 in FIG. 6.

The dosing estimation unit 1101 estimates, from information about a medicine acquired by the medicine information acquisition unit 602 and biological information acquired by the biological information acquisition unit 604, whether the medicine has been taken. The dosing estimation unit 1101 may recognize, from the information about the medicine, a time at which an effect of the medicine is estimated to be generated. When it is already recognized that the medicine has an effect on the user, the dosing estimation unit 1101 can estimate whether the medicine is taken in the effect generation time period with reference to biological information in the effect generation time period. Therefore, in a case of a medicine already established to have a certain effect on the user, the dosing estimation unit 1101 can provide an information processing device capable of supporting dosing of a medicine performed without being forgotten.

2

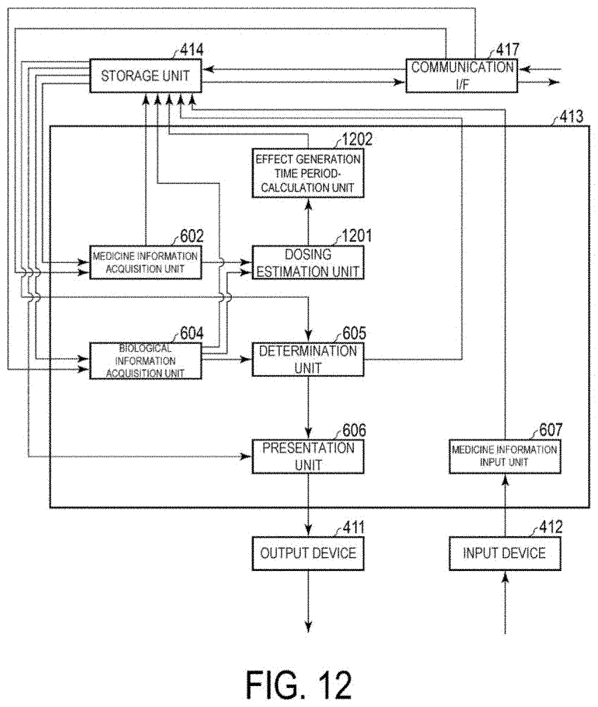

Next, another example of a modified example of the software configuration of the server device 170 according to the present embodiment will be described by using FIG. 12. The server device in the modified example is acquired by eliminating the dosing time acquisition unit 601 from the server device 170 in FIG. 6 and newly adding a dosing estimation unit 1201. An effect generation time period-calculation unit 1202 is slightly different in operation from the effect generation time period-calculation unit 603 illustrated in FIG. 6.

The dosing estimation unit 1201 estimates, from information about a medicine acquired by the medicine information acquisition unit 602 and biological information acquired by the biological information acquisition unit 604, time information about when the medicine has been taken. The dosing estimation unit 1201 can recognize, from the medicine information, information such as what kind of an effect and how long the effect is sustained after the medicine is taken. Furthermore, since the dosing estimation unit 1201 receives biological information from the biological information acquisition unit 604, the dosing estimation unit 1201 can estimate the time information about when the medicine has been taken by comparing this biological information with biological information when the medicine has been taken, which is included in the medicine information.

The effect generation time period-calculation unit 1202 tests biological information since a start of a medication time estimated by the dosing estimation unit 120, based on the medication time, and estimates a time period in which an effect of the medication appears in the biological information.

Therefore, according to the dosing estimation unit 1201 and the effect generation time period-calculation unit 1202, a time period in which an effect is generated by taking a medicine can be estimated.

3

### Hardware Configuration

### Blood Pressure Measurement Device

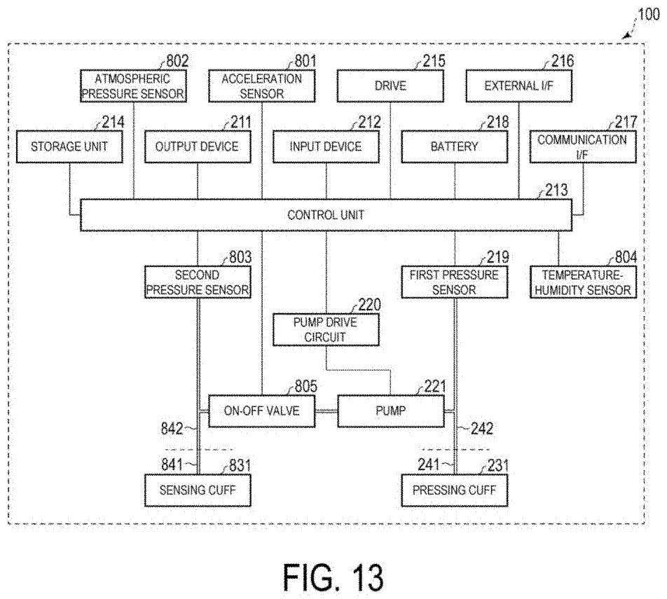

In the embodiment described above, as illustrated in FIG. 2, the blood pressure measurement device 100 includes the computer to which the output device 211, the input device 212, the control unit 213, the storage unit 214, the drive 215, the external interface 216, the communication interface 217, and the battery 218 are electrically connected. However, other devices for performing various types of information processing may be further provided. For example, as illustrated in FIG. 13, the blood pressure measurement device 100 may further include an acceleration sensor 801, an atmospheric pressure sensor 802, a second pressure sensor 803, a temperature-humidity sensor 804, an on-off valve 805, and a sensing cuff 831.

The acceleration sensor 801 is a 3-axis acceleration sensor. The acceleration sensor 801 outputs an acceleration signal representing acceleration in three directions orthogonal to one another to the control unit 213.

The atmospheric pressure sensor 802 detects atmospheric pressure. The atmospheric pressure sensor 802 outputs the atmospheric pressure data to the control unit 213.

The temperature-humidity sensor 804 measures ambient temperature and ambient humidity around the blood pressure measurement device 100, and outputs temperature and humidity data to the control unit 213.

Further, although not illustrated, the blood pressure measurement device 100 may include a GPS receiver.

The GPS receiver receives a GPS signal transmitted from each of a plurality of GPS satellites, and outputs the received GPS signals to the control unit 213. The control unit 213 calculates current position information about the blood pressure measurement device 100, namely, a position of a subject (user) wearing the blood pressure measurement device 100 by performing a distance measuring calculation based on each of the GPS signals described above.

Note that the blood pressure measurement device 100 may not necessarily include a distance measuring calculation function by the GPS receiver and the control unit 213. In this case, the blood pressure measurement device 100 acquires position information calculated by the server device 170 from the server device 170 via the communication interface 217.

Note that, in this case, the battery 218 supplies power to, for example, the output device 211, the control unit 213, the storage unit 214, the acceleration sensor 801, the atmospheric pressure sensor 802, the temperature-humidity sensor 804, the communication interface 217, the (first) pressure sensor 219, the second pressure sensor 803, the pump drive circuit 220, the pump 221, the on-off valve 805, and the GPS receiver (not illustrated).

The cuff structure 30 includes the curler 301, the pressing cuff 231, the back plate 303, and the sensing cuff 831.

The back plate 303 is disposed along the inner circumferential surface of the pressing cuff 231. The back plate 303 has a band shape. The back plate 303 is made of a resin, for example. The resin is polypropylene, for example. The back plate 303 functions as a reinforcing plate. Thus, the back plate 303 can transmit a pressing force from the pressing cuff 231 across the entire area of the sensing cuff 831.

The back plate 303 is interposed between the pressing cuff 231 and the sensing cuff 831, and extends along the Z direction. The sensing cuff 831 contacts a left wrist, and extends in the Z direction across an artery passing portion of the left wrist. The belt 20, the curler 301, the pressing cuff 231, and the back plate 303 act as a pressing member capable of generating a pressing force toward the left wrist, and compress the left wrist via the sensing cuff 831.

The on-off valve 805 is interposed in a second flow path formation member 842. The on-off valve 805 is, for example, a normally open solenoid valve. The opening and closing of the on-off valve 805 is controlled based on a control signal from the control unit 213. When the on-off valve 805 is in the open state, the pump 221 can supply fluid to the sensing cuff 831 via a flexible tube 841 and the second flow path formation member 842 that constitute a second flow path.

4

### Software Configuration

The blood pressure measurement device 100 according to the present embodiment may function as a computer further including an activity amount measurement unit, a step counting unit, a sleep state measurement unit, and an environment (temperature and humidity) measurement unit. The storage unit 214 stores a program (an activity amount measurement program, a step counting program, a sleep state measurement program, and an environment (temperature and humidity) measurement program) corresponding to each of the units, and develops a desired program into RAM when executing the necessary program. Then, the control unit 213 interprets and executes the program developed in the RAM by the CPU, and controls each component.

The blood pressure measurement device 100 may record biological information about the amount of activity, the number of steps, and/or a sleep state, instead of or in addition to time series data about a blood pressure value, for example.

The activity amount measurement unit detects acceleration by the acceleration sensor 801, and calculates the amount of activity. The activity amount measurement unit can calculate not only walking of the subject, but also the amount of activity in various activities such as household chores and desk work by using an acceleration signal. The amount of activity is, for example, an indicator associated with the activity of the subject, such as a walking distance, calorie consumption, or the amount of fat burned.

The step counting unit detects acceleration by the acceleration sensor 801 and atmospheric pressure by the atmospheric pressure sensor 802, and calculates the number of steps, the number of steps in fast waking, and the number of steps when going up stairs. Waking of the subject is calculated by using an acceleration signal. The step counting unit can calculate the number of steps of the subject, the number of steps in fast waking, the number of steps when going up stairs, and the like by using atmospheric pressure data and an acceleration signal.

The sleep state measurement unit can estimate a sleep state by detecting acceleration by the acceleration sensor 801 and detecting a state of tossing and turning by an acceleration signal.

The environment (temperature and humidity) measurement unit associates environmental data indicating environmental temperature and environmental humidity measured by the temperature-humidity sensor 804 with a measurement time in the temperature-humidity sensor 804, and stores the environmental data in the storage unit 214. A temperature (change in temperature) is considered as one of elements that may cause blood pressure fluctuations of a human being, for example. Thus, the environmental data are information that may be a factor of blood pressure fluctuations of the subject.

5

### Dosing Time and Medicine Information

A dosing time in the dosing time reception unit 502 and/or the dosing time acquisition unit 601 may be automatically set, based on medicine information from the server device 170 and a medicine record by an application that is installed on the mobile information terminal 150 and manages dosing information, and may be registered in the blood pressure measurement device 100. More specifically, the server device 170 acquires medicine information about a medicine from a network or the like, and the mobile information terminal 150 refers to a dosing record of the medicine corresponding to the medicine information, sets a dosing time of the medicine, and registers the dosing time in the blood pressure measurement device 100. Then, the blood pressure measurement device 100 starts measuring biological information, based on the dosing time.

Further, simply, a dosing time included in medicine information from the server device 170 may be registered in the blood pressure measurement device 100, and a measurement of biological information may start at the dosing time. Further, instead of the server device 170 acquiring medicine information, the mobile information terminal 150 may acquire the medicine information from a specific code (for example, a QR code (registered trademark)) that uniquely identifies the medicine information, and start a measurement of biological information at a dosing time included in the medicine information.

As medicine information in the medicine information input unit 607, medicine information acquired by the server device 170 described above from the network may be simply used as it is, or medicine information acquired by the mobile information terminal 150 described above from a specific code may be simply used as it is.

In addition, a meal time of the user may be acquired from an application that manages meal information in the mobile information terminal 150, and a dosing time may be set based on the meal time and registered in the blood pressure measurement device 100. In this case, the meal time of the user is calculated by a predetermined algorithm, and, for example, statistics are taken every day of the week and an average meal time is set for each of three meals. Various modifications are conceivable for the algorithm, which is not particularly limited.

6

When the operation unit 107 included in the input device 212 is pressed (turned on), the blood pressure measurement device 100 may start a blood pressure measurement (perform the operation in FIG. 10).

7

In the embodiment described above, a blood pressure value of the user is measured by an oscillometric method by using the cuff structure 30. However, a case where only a blood pressure value is measured is not limited to this. For example, a pressure pulse wave sensor that detects a pressure pulse wave for each heartbeat may be provided, and may detect a pressure pulse wave of a radial artery running through a target measurement site (for example, a left wrist) and measure a blood pressure value (systolic blood pressure value and diastolic blood pressure value) (tonometry method). The pressure pulse sensor may detect a pulse wave of the radial artery running through the target measurement site (for example, the left wrist) as a change in impedance, and measure a blood pressure value (impedance method). The pressure pulse wave sensor may include a light emitting element that irradiates light at an artery running through a corresponding portion of the target measurement site and a light receiving element that receives the reflected light (or transmitted light) of the light, and a blood pressure value may be measured by detecting a pulse wave of the artery as a change in volume (photoelectric method).

Further, the pressure pulse wave sensor may include a piezoelectric sensor that is in contact with the target measurement site, and a blood pressure value may be measured by detecting a strain due to pressure of the artery running through the corresponding portion of the target measurement site as a change in electric resistance (piezoelectric method). Furthermore, the pressure pulse wave sensor may include a transmission element that sends an electromagnetic wave (transmission wave) to the artery running through the corresponding portion of the target measurement site and a reception element that receives the reflected wave of the electromagnetic wave, and a blood pressure value may be measured by detecting a change in distance between the artery and the sensor due to the pulse wave of the artery as a phase shift between the transmission wave and the reflected wave (electromagnetic wave irradiation method). Note that, when a physical quantity with which a blood pressure value can be calculated can be observed, a method other than these methods may be applied.

8

The device of the present invention can also be achieved by a computer and a program, and it is also possible to record the program in a recording medium (or storage medium) or to provide the program through a network.

Further, each of the devices and portions of the devices described above can be implemented in both of a hardware configuration and a combined configuration of a hardware resource and software. The software of the combined configuration is previously installed on a computer from a network or a computer readable recording medium (or storage medium) and executed by a processor of the computer, and thus a program for achieving a function of each of the devices on the computer is used.

Note that the present invention is not limited to the above-described embodiments, and the components can be modified and embodied in an implementation stage without departing from the gist thereof. Further, various types of the invention can be formed by an appropriate combination of the plurality of components disclosed in the above-described embodiments. For example, several components may be deleted from all the components represented in the embodiment. Furthermore, the components in different embodiments may be appropriately combined.

Further, “and/or” means any one or more items among items connected with “and/or” and listed. For a specific example, “x and/or y” means any element of a set {(x), (y), (x, y)} formed of three elements. For another specific example, “x, y, and/or z” means any element of a set {(x), (y), (z), (x, y), (x, z), (y, z), (x, y, z)} formed of seven elements.

9

Further, some or all of the embodiments described above may also be described as supplementary notes below, which are not limited thereto.

### Supplementary Note 1

An information processing device including a hardware processor and a memory, wherein

the hardware processor is configured to

acquire information about a medicine and time information about when the medicine is to be taken,

calculate an effect generation time period in which an effect of the medicine is estimated to be generated, based on the information about the medicine and the time information, and

perform processing for acquiring information about a living body, based on the effect generation time period, and

the memory includes a storage unit configured to store the effect generation time period.

### Supplementary Note 2

An information processing device including a hardware processor and a memory, wherein

the hardware processor is configured to

acquire information about a medicine and information about a living body,

estimate, from the information about the medicine and the information about the living body, time information about when the medicine has been taken,

calculate an effect generation time period in which an effect of the medicine is estimated to be generated, based on the information about the medicine and the time information, and

perform processing for acquiring information about a living body, based on the effect generation time period, and

the memory includes a storage unit configured to store the effect generation time period.

### Supplementary Note 3

An information processing method, including:

acquiring, by using at least one hardware processor, information about a medicine and time information about when the medicine is to be taken;

calculating an effect generation time period in which an effect of the medicine is estimated to be generated, based on the information about the medicine and the time information, by using at least one hardware processor; and

performing processing for acquiring information about a living body, based on the effect generation time period, by using at least one hardware processor.

### Supplementary Note 4

An information processing method, including:

acquiring information about a medicine and information about a living body by using at least one hardware processor;

estimating, by using at least one hardware processor and from the information about the medicine and the information about the living body, time information about when the medicine is estimated to be taken;

calculating an effect generation time period in which an effect of the medicine is estimated to be generated, based on the information about the medicine and the time information, by using at least one hardware processor; and

performing processing for acquiring information about a living body, based on the effect generation time period, by using at least one hardware processor.

### REFERENCE SIGNS LIST

10 Body
10A Case, 10B Glass, 10C Back lid
20 Belt
30 Cuff structure, 30a First end, 30b Second end
100 Blood pressure measurement device
101 Blood pressure measurement unit
102 Storage unit
103 Transmission unit
104 Reception unit
105 Operation unit
106 Timing unit
107 Operation unit
150 Mobile information terminal
170 Server device
171 Acquisition unit
172 Calculation unit
173 Storage unit
174 Determination unit
175 Presentation unit
176 Transmission unit
177 Timing unit
201 Belt portion
201a Root portion, 201b Tip portion
202 Belt portion, 202a Root portion, 202b Tip portion, 202c Small hole
203 Buckle, 203A Frame body, 203B Prong, 203C Connecting rod
204 Belt holding portion
211 Output device
212 Input device
213 Control unit
214 Storage unit
215 Drive
216 External interface
217 Communication interface
218 Battery
219 Pressure sensor
220 Pump drive circuit
221 Pump
231 Pressing cuff
241 Flexible tube
242 Flow path formation member
301 Curler, 303 Back plate
401 Connecting rod, 402 Connecting rod
411 Output device
412 Input device
413 Control unit
414 Storage unit
415 Drive
416 External interface
417 Communication interface
418 Power source device
501 Presentation unit
502 Dosing time reception unit
503 Effect generation time period-reception unit
504 Blood pressure measurement unit
601 Dosing time acquisition unit
602 Medicine information acquisition unit
603 Effect generation time period-calculation unit
604 Biological information acquisition unit
605 Determination unit
606 Presentation unit
607 Medicine information input unit
801 Acceleration sensor
802 Atmospheric pressure sensor
803 Pressure sensor
804 Temperature-humidity sensor
805 On-off valve
831 Sensing cuff
841 Flexible tube
842 Flow path formation member
1101, 1201 Dosing estimation unit
1202 Effect generation time period-calculation unit.

1. An information processing device, comprising:
a processor; and
a memory, wherein
the processor is configured to acquire information about a medicine and time information about when the medicine is to be taken,
the processor is configured to calculate an effect generation time period in which an effect of the medicine is estimated to be generated, based on the information about the medicine and the time information, and
the processor is configured to perform processing for acquiring information about a living body, based on the effect generation time period.

2. The information processing device according to claim 1, wherein
the processor is configured to determine whether the medicine is effective on the living body, based on the information about the living body.

3. The information processing device according to claim 1, wherein
the processor is configured to estimate, from the information about the medicine and the information about the living body, whether the medicine has been taken.

4. An information processing device, comprising:
a processor; and
a memory, wherein
the processor is configured to acquire information about a medicine and information about a living body,
the processor is configured to estimate, from the information about the medicine and the information about the living body, time information about when the medicine has been taken,
the processor is configured to calculate an effect generation time period in which an effect of the medicine is estimated to be generated, based on the information about the medicine and the time information, and
the processor is configured to perform processing for acquiring information about a living body, based on the effect generation time period.

5. The information processing device according to claim 4, wherein
the processor is configured to determine whether the medicine is effective on the living body, based on the information about the living body.

6. The information processing device according to claim 2, wherein
the processor is configured to determine whether the medicine is effective on the living body by comparing first biological information measured at a time included in the time information with second biological information measured in the effect generation time period.

7. The information processing device according to claim 6, wherein
the processor is configured to compare a first value indicated by the first biological information with a second value indicated by the second biological information, and make a determination from a sign of a difference between the first value and the second value and a comparison result between an absolute value of the difference and a preset effective threshold value.

8. The information processing device according to claim 2, wherein
the processor is configured to present as candidates for medication, when the processor determines that the medicine is not effective, one or more medicines from a plurality of medicines including the medicine expected to have an effect.

9. The information processing device according to claim 8, wherein
the memory is configured to store the information about the medicine, the effect generation time period, and the information about the living body in association with one another, and
the processor is configured to present a type and a quantity of a medicine expected to have an effect, based on information stored in the memory.

10. The information processing device according to claim 1, wherein
the information about the medicine includes a type of a medicine, an effect of a medicine, a use of a medicine, and a side effect of a medicine.

11. The information processing device according to claim 1, wherein
the information about the living body includes a blood pressure value or a pulse.

12. A biological information measuring device, comprising:
a processor; and
a memory, wherein
the processor is configured to perform processing for measuring information about a living body, based on time information about when a medicine is to be taken based on information about the medicine, and an effect generation time period in which an effect of the medicine is estimated to be generated, and
the processor is configured to transmit the information about the living body.

13. An information processing method, comprising:
acquiring information about a medicine and time information about when the medicine is to be taken;
calculating an effect generation time period in which an effect of the medicine is estimated to be generated, based on the information about the medicine and the time information; and
performing processing for acquiring information about a living body, based on the effect generation time period.

14. An information processing method, comprising:
acquiring information about a medicine and information about a living body;
estimating, from the information about the medicine and the information about the living body, time information about when the medicine has been taken;
calculating an effect generation time period in which an effect of the medicine is estimated to be generated, based on the information about the medicine and the time information; and
performing processing for acquiring information about a living body, based on the effect generation time period.

15. A non-transitory recording medium in which a program for causing the processor included in the information processing device according to claim 1 to acquire, calculate and perform processing.

16. The information processing device according to claim 2, wherein
the processor is configured to estimate, from the information about the medicine and the information about the living body, whether the medicine has been taken.

17. The information processing device according to claim 5, wherein
the processor is configured to determine whether the medicine is effective on the living body by comparing first biological information measured at a time included in the time information with second biological information measured in the effect generation time period.

18. The information processing device according to claim 5, wherein
the processor is configured to present as candidates for medication, when the processor determines that the medicine is not effective, one or more medicines from a plurality of medicines including the medicine expected to have an effect.

19. The information processing device according to claim 6, wherein
the processor is configured to present as candidates for medication, when the processor determines that the medicine is not effective, one or more medicines from a plurality of medicines including the medicine expected to have an effect.

20. The information processing device according to claim 7, wherein
the processor is configured to present as candidates for medication, when the processor determines that the medicine is not effective, one or more medicines from a plurality of medicines including the medicine expected to have an effect.
